(19) United States
(12) Patent Application Publication
(10) Pub. No.: US 2021/0008081 A1
(43) Pub. Date: Jan. 14, 2021

(54) ORAL LIQUID FORMULATIONS OF ABIRATERONE

(71) Applicant: LEIUTIS PHARMACEUTICALS PVT, LTD, HYDERABAD (IN)

(72) Inventors: Chandrashekhar KOCHERLAKOTA, Hyderabad (IN); Nagaraju BANDA, Hyderabad (IN); Santhosh Kumar MANKALA, Hyderabad (IN)

(21) Appl. No.: 17/042,351

(22) PCT Filed: Mar. 28, 2019
(86) PCT No.: PCT/IB2019/052528

§ 371 Date: Sep. 28, 2020

(30) Foreign Application Priority Data
Mar. 29, 2018 (IN) 201841011872

(51) Int. Cl. A61K 31/58; A61K 9/00; A61K 9/107; A61K 47/26; A61K 47/24

(52) U.S. Cl. CPC A61K 31/58; A61K 9/0053; A61K 47/24; A61K 47/26; A61K 9/107

(57) ABSTRACT

Liquid formulations of Abiraterone intended for oral administration are provided. Also provided are suspension and emulsion formulations of Abiraterone and processes of preparing such formulations. Further, embodiments of the invention relates to Nanoemulsion formulations of Abiraterone including Abiraterone, oil phase, aqueous phase, one or more emulsifying agents and optionally other pharmaceutical excipients, wherein the concentration of excipients are optimized for better stability.

### CROSS-REFERENCE TO RELATED APPLICATIONS

This application claims priority to PCT Application No. PCT/M2019/052528, having a filing date of Mar. 28, 2019, based on IN 201841011872, having a filing date of Mar. 29, 2018, the entire contents both of which are hereby incorporated by reference.

### BACKGROUND

Abiraterone acetate is a selective inhibitor of 17α-hydroxylase and C17,20-lyase enzymatic activities of cytochrome P450 (CYP17). Abiraterone is used in combination with prednisone in metastatic castration-resistant prostate cancer and metastatic high-risk castration-sensitive prostate cancer. Abiraterone acetate is a prodrug of Abiraterone and is rapidly deacetylated to Abiraterone in vivo.

U.S Pat. No. 5,604,213 to Barrie et al., describes Abiraterone acetate and related analogs and the process of preparation.

WO 2013/012959 to Casebier et al., discloses a composition comprising a solid dispersion of Abiraterone and a solid matrix, wherein Abiraterone is dispersed in the solid matrix.

WO 2013/164473 to Grenier et al., discloses Abiraterone acetate dissolved or dispersed in a carrier, wherein the carrier comprises one or more lipid excipients.

WO 2014/145813 to Bosch et al., discloses a method of producing a composition comprising nanoparticles of Abiraterone acetate. The pharmaceutical composition disclosed can be a unit dosage form such as a capsule or tablet.

U.S Pat. No. 9,511,078 to Dipen et al., discloses pharmaceutical formulation containing a low aqueous solubility drug, such as Abiraterone formulated in a capsule, wherein the drug is solubilized within the other components of the pharmaceutical formulation which forms a nanoemulsion upon exposure to an aqueous environment, such as the gastrointestinal tract.

Abiraterone acetate is classified as class IV compound (low solubility low permeability) according to the biopharmaceutical classification system. Abiraterone acetate is practically insoluble in aqueous media over a wide range of pH and sparingly soluble to freely soluble in organic solvents, leading to low bioavailability of Abiraterone acetate for Zytiga®. The absolute bioavailability is not known, although the bioavailability from the commercial tablet in the fasted state is unlikely to be higher than 10%. Hence to achieve the desired therapeutic effect, the recommended dose of 1000 mg is administered.

The recommended dose of ZYTIGA® is 1,000 mg (two 500 mg tablets or four 250 mg tablets) orally once daily with prednisone 5 mg. 4 tablets or 2 tablets (1,000 mg) of Abiraterone have to be swallowed whole with water on an empty stomach, either one hour before or two hours after a meal.

It is often advantageous to have an oral liquid formulation as it provides ease of administration to the patient. The conventional art cited herein teaches various methods to improve the bioavailability but do not disclose liquid formulations of Abiraterone with improved bioavailability.

The following addresses the disadvantages associated with the conventional art and provides stable oral liquid formulations of Abiraterone.

### SUMMARY

An aspect relates to liquid formulations of Abiraterone intended for oral administration.

One aspect of embodiments of the invention provides suspension and emulsion formulations of

Abiraterone and process of preparing the formulations.

Another aspect of embodiments of the invention provides nano-suspension and nano-emulsion formulations of Abiraterone and process of preparing the formulations.

Yet another aspect of embodiments of the present invention provides oral liquid formulations of Abiraterone, wherein the dose volume of the liquid formulation is less than 120 ml.

### DETAILED DESCRIPTION

As used herein “Abiraterone” refers to the pharmaceutically acceptable derivatives such as salts, solvates, hydrates, polymorphs and prodrugs thereof, of Abiraterone or Abiraterone acetate.

The term “stable” means the formulations which remain stable during the entire shelf-life of the formulation. The stable formulations of embodiments of the invention retain an assay value of at least 90% throughout the shelf life.

The term “liquid formulation” includes suspensions and emulsions intended for oral administration.

The term “emulsion” can be defined as a system including a continuous phase and a dispersed phase, wherein the dispersed droplets are present in the continuous phase. According to embodiments of the invention emulsion also includes nano-emulsion.

The term “suspension” can be defined as dispersion in which insoluble solid particles are dispersed in a liquid medium. According to embodiments of the invention suspension also includes nano-suspension.

As used herein, “dose volume” is defined as the volume which is to be administered orally once to a patient to produce required therapeutic action.

As used herein “ready to use” liquid formulation refers to formulations that doesn't require further dilution or reconstitution and are intended to be used as such.

As used here the term “median diameter (D50)” is defined as the equivalent diameter where 50% of the particles in the formulation lie below the value.

As used herein “average globule/droplet size” refers to the z-average of globule, which is average particle diameter of the globules.

The globule size or particle size are determined by any of the known instruments. The instrument used here is LITESIZER™-500.

Abiraterone acetate is practically insoluble in water, which is one of the factors leading to low bioavailability of ZYTIGA®. In order to achieve the desired therapeutic effect, the recommended dose of 1000 mg is administered as four tablets of 250 mg each or two tablets of 500 mg each. This situation is unsatisfactory and inconvenient to the patients, particularly in cases where medications usually consist of multiple drug regimen demanding the administration of large number of tablets or capsules. This often leads to difficulty in swallowing.

Abitraterone acetate is indicated to be administered twice daily. The patients are instructed not to take any food for at least two hours before the administration of the tablets or for at least one hour after the administration of the tablets. This creates a lot of discomfort to the patients, particularly to those associated with metabolic disorders. Embodiments of the present invention of liquid oral formulation overcome these disadvantages. The liquid oral formulations like suspensions and emulsions with specific globule size are expected to yield better and uniform bioavailability.

Liquid formulations of Abiraterone prepared according to embodiments of the invention can overcome the disadvantages of conventional art and improve patient compliance. It is also convenient for dose adjustments and ease of administration.

The USFDA recommends dosage adjustment in case of hepatotoxicity and co-administration of CYP3A4 inducers. Unlike the commercially marketed tablet formulations, the dose adjustment can be easily done with liquid formulations prepared according to embodiments of the invention. A corresponding volume of the liquid formulation may be administered depending on the dose adjustment required. Examples of liquid formulations of embodiments of the invention include, but are not limited to, suspensions, emulsions, nano-suspensions, and nano-emulsions.

Another advantage of embodiments of the invention relates to the dose volume of Abiraterone to be administered. The dose volume to be administered is less than 120 ml and can be ingested easily. In some embodiments, the dose volume ranges from about 0.5 ml to 60 ml. The concentration of Abiraterone in the oral liquid formulations ranges from about 5 mg/ml to 500 mg/ml.

One embodiment of the invention relates to liquid formulations of Abiraterone intended for oral administration. These may be in the form of suspensions or emulsions.

Another embodiment of the invention relates to suspension and emulsion formulations of Abiraterone comprising
(i) abiraterone,
(ii) suspending agent or emulsifying agent,
(iii) one or more solvents,
(iv) other pharmaceutically acceptable excipients, wherein the formulation is a ready to use oral liquid.

To overcome the disadvantages of the conventional art, inventors of the embodiments of the present invention developed formulations which exhibit improved absorption thereby improving the bioavailability of the drug. Nano-suspension and nano-emulsion formulations prepared according to embodiments of the invention exhibit improved absorption, thus increasing the availability of the drug in the body. As a result, dose reduction may be achieved resulting in cost effective formulations with reduced side effects.

Nano-Suspensions of Abiraterone

Nano-suspension formulations of Abiraterone are prepared by micronization of the drug using suitable techniques, thus increasing the dissolution rate of the drug thereby improving the bioavailability of Abiraterone. Nano-suspension formulations prepared according to embodiments of the invention exhibit rapid onset of action, reduced variability of the drug in fasted and fed states and improved bioavailability.

Embodiments of the present invention provide nano-suspension formulations of Abiraterone and process of preparing such formulations. The median diameter (D50) of Abiraterone in nano-suspension formulation is less than 600 nm.

Another embodiment of the invention relates to nano-suspension formulations of Abiraterone comprising:
(i) abiraterone,
(ii) one or more suspending agents,
(iii) one or more wetting agents,
(iv) one or more solvents, and
(v) optionally other pharmaceutically acceptable excipients, wherein the median diameter (D50) of Abiraterone is less than 600 nm.

The concentration of the excipients may be adequately selected and adjusted along with the process parameters to avoid stability issues such as agglomeration or cluster formation and also to attain and maintain the desired particle size.

Nano-suspension formulations prepared according to embodiments of the invention comprise suspending agents, wetting agents, solvents and other pharmaceutically acceptable excipients. Other pharmaceutically acceptable excipients can be selected from stabilizing agents, dispersing agents, thickening agents, chelating agents, buffering agents, preservatives, sweetening agents, flavoring agents, coloring agents, permeation stabilizers and the like.

Suspending agents are added to prevent sedimentation by affecting the rheological behavior of a suspension and thus reduces the movement (sedimentation) of suspended particles and physically stabilizes the product. Suspending agents include, but not limited to cellulosic derivatives, polysaccharides, gums, carboxyvinyl polymers and synthetic polymers such as methyl cellulose, ethyl cellulose, carboxy methyl cellulose, hydroxy ethyl cellulose, hydroxy propyl ethyl cellulose, crosslinked polyacrylic acid polymer, polyvinylpyrrolidone (PVP), poloxamers, carbopol, polyvinyl alcohol, xanthan gum, guar gum, tragacanth, acacia, gelatin, carrageenan, agar-agar, alginic acid, sodium alginate, propylene glycol alginate, carbomer, magnesium aluminium silicate, hydroxypropyl methylcellulose, hydroxypropylcellulose, microcrystalline cellulose, polydextrose, sucrose, sorbitol, xylitol, dextrose, fructose, maltitol, bentonite and the like.

The use of wetting agents allows removing the air from the surface and easy penetration of the vehicle into the pores and disperse solids in continuous liquid phase. Wetting agents are selected from the group comprising surfactants, alcohol, glycerin, propylene glycol and other polar liquids. Surfactants lower the surface tension of a liquid allowing easier spreading and lower the interfacial tension between two liquids. Surfactants are selected from the group comprising anionic, cationic and non-ionic. The examples include, but are not limited to, alkyl poly(ethylene oxide), copolymers of poly(ethylene oxide) and poly(propylene oxide), alkyl polyglucosides, polysorbates, such as tween 20, tween 80; and dodecyl dimethylamine oxide, sodium dodecyl sulfate, ammonium lauryl sulfate, sodium lauryl ether sulfate, sodium lauryl sulfate (SLS), cetyl trimethylammonium bromide, cetylpyridinium chloride, benzethonium chloride and labrasol.

Permeation stabilizer selected from Vitamin E TPGS. Suitable solvents selected from water.

The nano-suspensions of embodiments of the invention may be produced by any method conventionally employed to produce nano-suspensions. These methods include precipitation, high pressure homogenization and milling. In some embodiments, such nano-suspensions may be produced via high pressure homogenization. This technique typically involves a three step process in which a powder is dispersed in a solution to form a pre suspension; the pre suspension is homogenized employing a high shear homogenizer; and is then homogenized at high pressure using high pressure homogenizer until the nano-suspension is formed with the desired particle size. Process parameters are optimized to achieve a desired particle size and to obtain a stable nano-suspension formulation.

Nano Emulsion of Abiraterone

One embodiment of the invention provides stable nano-emulsion formulation, wherein the average globule size is less than 800 nm.

Another embodiment of the invention provides nanoemulsion formulations of Abiraterone comprising:
(i) abiraterone,
(ii) oil phase,
(iii) one or more emulsifying agents,
(iv) aqueous phase,
(v) optionally other pharmaceutically acceptable excipients, wherein the average globule size is less than 800 nm.

There are many challenges associated with developing nanoemulsion formulations. In the case of Abiraterone, it is even more so because of the high lipophilicity of the drug. The studies were focused on maintaining the stability of the formulation, achieving the ideal globule size, maintaining the globule size in accelerated stability studies and recommended storage conditions, optimizing the process parameters to achieve the globule size, the role of excipients and their concentration on the stability of the nano emulsion.

An essential component of the nanoemulsion system is an oil phase comprising individual oil droplets, which represent the internal hydrophobic or oil phase. The oil component is selected based on the solubility of the abiraterone in the oil. The oil may be a single entity or mixture. Selection of suitable oil in the correct proportions along with other pharmaceutically acceptable excipients is important to obtain a stable product.

Suitable oils for use in the preparation of nano emulsion include, vegetable oils, medium chain triglycerides (MCT's), soya bean oil, corn oil, safflower oil, sunflower oil, castor oil, olive oil, monoesters of glycerin having fatty acid groups comprising about 8 to 12 carbon atoms, monoesters of propylene glycol having fatty acid groups comprising about 8 to 12 carbon atoms, long chain triglycerides (usually about 14 to 22 carbons in length), glyceryl caprylate, glyceryl caprate, glyceryl monolaurate, propylene glycol monocaprylate, glyceryl monocaprylate/monocaprate, propylene glycol monolaurate, including medium chain fatty acids (i.e., caprylic acid, capric acid, lauric acid) and combinations.

The nano-emulsion formulation comprises one or more emulsifying agents or surfactants. Suitable emulsifying agents are selected from the group, but not limited to polyethoxylated oils, such as polyoxyl castor oils (e.g., PEG-40 hydrogenated castor oil, polyoxyl 35 castor oil, etc.), surfactants, polysorbates, sorbitan esters of fatty acids such as sorbitan monolaurate, sorbitan monopalmitate, sorbitan monostearate, sorbitan monooleate and phospholipids such as lecithin, egg lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin. In some embodiments, emulsifying agents may be polysorbates and lecithin.

Aqueous phase of the nano-emulsion formulation includes water.

Nano-emulsion formulation of embodiments of the invention comprises oil phase, aqueous phase, emulsifying agents and other pharmaceutically acceptable excipients selected from carriers, stabilizing agents, chelating agents, buffering agents, preservatives, sweetening agents, flavoring agents, coloring agents and the like.

The formulation of embodiments of the present invention comprises stabilizing agents such as sugars and amino acids. Suitable stabilizers include glucose, trehalose, sucrose, mannitol, sorbitol, arginine, glycine, proline, methionine, lysine and the like.

Suitable chelating agents include, but not limited to DOTA (1,4,7,10-tetraazacyclododecane-1,4,7,10-tetraacetic acid), DTPA (diethylene triaminepentaacetic acid), EDTA (ethylenediaminetetraacetic acid) and the like.

Buffering agents are used to maintain the original acidity or basicity of a composition. Suitable buffering agents include, but are not limited to citric acid, sodium citrate, sodium phosphate, potassium citrate and mixtures thereof.

Suitable preservatives include sodium benzoate, benzoic acid, sorbic acid, benzethonium chloride, benzalkonium chloride, parabens, thiomersal, sodium propionate, chlorhexidine, chlorobutanol, chlorocresol, cresol, phenol, phenylmercuric salts, potassium sorbate, sodium bisulfite, sodium metabisulfite, Butylated hydroxy toluene, sodium salts of hydroxybenzoate and the like.

Sweetening agents selected from the group comprising of maltitol, sucrose, dextrose, fructose, glucose, inulin, isomalt, lactitol, maltose, mannitol, sucralose, trehalose, xylitol, sorbitol, saccharin, thaumatin, sodium cyclamate, acesulfame potassium, aspartame and the like.

Flavouring agents selected from, but not limited to peppermint flavour, mint flavour, orange flavour, lemon flavor, grape flavor, strawberry flavor, artificial cream flavor, vanilla, cherry, raspberry, Masking 2521, Pineapple flavor and the like.

Suitable coloring agents include tartrazine.chrysoine, quinoline yellow, carminic acid, carmoisine, amaranth, betanin, titanium dioxide, iron oxides and hydroxides and the like.

Maintaining the globule size is very important for the nanoemulsion formulations. The inventors carried out various experiments for the selection of emulsifying agents and optimizing their concentrations to arrive at the target globule size. The ideal globule size may be less than 800 nm, and may be below 400 nm and or below 300 nm in some embodiments.

TABLE 1
Effect of concentration of surfactants

| Ingredients | Qty (% w/v) for 250 mg/10 ml concentration | |
|---|---|---|
| Abiraterone Acetate | 2.500 | 2.500 |
| Medium chain triglycerides | 30.000 | 30.000 |
| Butylated hydroxy Toluene | 0.075 | 0.075 |
| Sodium meta bisulfate | 0.300 | 0.300 |
| Sorbic acid | 0.060 | 0.060 |
| Polysorbate 80 | 2.400 | 1.800 |
| Lecithin | — | 0.600 |
| Water | 64.478 | 64.478 |
| EDTA | 0.038 | 0.038 |
| Masking 2521 | 0.075 | 0.075 |
| Saccharine | 0.075 | 0.075 |
| | 100.00 | 100.00 |

The stability details of the above formulations when stored for 3months at various conditions are tabulated below:

TABLE 2
Stability data of above formulation

| | 25° c. | | 30° c. | | 40° c. | |
|---|---|---|---|---|---|---|
| | | With | | With | | With |
| | With | polysorbate + | With | polysorbate + | With | polysorbate + |
| Parameter | polysorbate | lecithin | polysorbate | lecithin | polysorbate | lecithin |
| Globule | 213.69 | 205.61 | 216.74 | 197.55 | 221.91 | 202.12 |
| size (nm) | *(237.2 nm) | *(195.94) | | | | |
| Polydisperse | 8.4 | 9.0 | 16.5 | 13.4 | 15.6 | 21.5 |
| index | | | | | | |
| (%) | | | | | | |
| D10 (nm) | 137.475 | 135.243 | 137.539 | 125.991 | 141.044 | 126.104 |
| D50 (nm) | 193.918 | 181.501 | 192.808 | 181.764 | 199.18 | 179.01 |
| D90 (nm) | 276.833 | 245.738 | 270.481 | 264.004 | 283.095 | 253.315 |
| Zeta | −8 | 4.01 | −4.7 | 4.26 | −4.1 | 4.17 |
| potential | | | | | | |
| (mV) | | | | | | |
| Viscosity | 3.68 | 0.983 | 3.12 | 0.982 | 3.87 | 0.984 |
| (mPa · s) | | | | | | |
| Density | 0.982 | −25.3 | 0.984 | −22.8 | 0.984 | −21.5 |
| (mg/mL) | | | | | | |

(*initial values)

From the above data, it is clear that polysorbate and a combination of polysorbate and lecithin are able to maintain the globule size even at accelerated temperatures.

The nano emulsion of embodiments of the invention can be made by the following steps:
(i) preparation of oil phase, dissolve abiraterone acetate in oil phase
(ii) preparation of aqueous phase
(iii) stirring and mixing of both phases
(iv) high shear homogenization, once or more
(v) high pressure homogenization

Embodiments of the present invention are further illustrated by the following examples which are provided merely to be exemplary of embodiments of the invention and do not limit embodiments of the scope of the invention. Certain modifications and equivalents will be apparent to those skilled in the art and are intended to be included within the scope of embodiments of the present invention.

### Example 1

### Suspension Formulation

| S. No | Ingredients | Quantity (mg) |
|---|---|---|
| 1. | Abiraterone acetate | 1000 |
| 2. | Medium chain triglyceride | 1000 |
| 3. | Polyvinyl pyrrolidone | 40 |
| 4. | Carbopol | 10 |
| 5. | Vanilla flavor | 16 |
| 6. | Saccharine | 8 |
| 7. | Sodium lauryl sulphate | 45 |
| 8. | Vitamin E-TPGS | 25 |
| 9. | Sodium hydroxide | q.s |
| 10. | Water | q.s |

* q.s: Quantity sufficient

### Manufacturing Process:

1. Polyvinyl pyrrolidone (PVP), sodium lauryl sulphate, saccharin and vanilla flavor were added to some amount of water and stirred (Phase A).
2. Vitamin E-TPGS was added to some amount of water, then it was heated with simultaneous stirring till vitamin E-TPGS was completely dissolved. (Phase B)
3. Abiraterone acetate was added to medium chain triglyceride and stirred (Phase C).
4. Carbopol was added to required quantity of water and stirred till a clear dispersion was obtained. Sodium hydroxide was added and stirred till the fluid becomes thick as a transparent and translucent gel. (Phase D)
5. Phase A was transferred to phase B with stirring till a homogenous mixture was obtained.
6. Homogenous mixture obtained in step 5, was added to phase C with high speed stirring till the phase becomes uniform dispersion.
7. The obtained dispersion of step 6, was transferred to phase D and the product was homogenized using suitable equipment.

Suspension formulation prepared according to embodiments of the invention was subjected to stability study. The formulations prepared were stored at room temperature (25° C.±2° C./60±5%RH) and at accelerated temperature (40° C.±2° C./75±5%RH) for a period of 6 months (6M). The stability data is summarized in table 3.

TABLE 3
Stability data of Abiraterone suspension formulation
Stability data of abiraterone suspension formulation
prepared according to example 1

| | | 1 M; | 6 M; | 1 M; | 6 M; |
|---|---|---|---|---|---|
| Condition | Initial | 25° C. | 25° C. | 40° C. | 40° C. |
| Total impurities (% w/w) | 1.05 | 1.15 | 0.90 | 1.20 | 1.79 |
| Assay (%) | 101.2 | 101.5 | 101.3 | 101.2 | 100.8 |

*1 M: 1 Month; 6 M: 6 Months

### Example 2

### Nano-Emulsion Formulation

| S. No | Ingredients | Quantity (mg) |
|---|---|---|
| 1. | Abiraterone acetate | 250.0 |
| 2. | Medium chain triglyceride | 4000.0 |
| 3. | Sorbic acid | 12.0 |
| 4. | Polysorbate 80 | 480.0 |
| 5. | EDTA | 7.5 |
| 6. | Flavouring agent | 15.0 |
| 7. | Saccharine | 15.0 |
| 8. | Purified Water | q.s |

Manufacturing Process:
1. Required quantity of the medium chain triglyceride was taken in a suitable vessel and heated to 65 to 70° C. Abiraterone acetate was added and temperature was maintained till abiraterone was completely dissolved. (Oil phase)
2. Sorbic acid, polysorbate 80, EDTA, saccharine and flavouring agent were added to required quantity of water (aqueous phase) and heated to 65 to 70° C. till all ingredients were dissolved.
3. The oil phase was added to aqueous phase with simultaneous homogenization at 5000 rpm using high shear homogenizer till a coarse emulsion was obtained.
4. The coarse emulsion was further homogenized at 5000 psi over high pressure homogenizer for 3 passes maintaining the product temperature constant at 65 to 70° C.

### Example 3

### Nano-Emulsion Formulation

| S. No | Ingredients | Quantity (mg) |
|---|---|---|
| 1. | Abiraterone acetate | 500 |
| 2. | Medium chain triglyceride | 4000 |
| | (Labrafac lipophile WL 1349) | |
| 3. | Butylated hydroxy toluene | 3.75 |
| 4. | Sodium metabisulfate | 15 |
| 5. | Sorbic acid | 12 |
| 6. | Polysorbate 80 | 480 |
| 7. | EDTA | 7.5 |
| 8. | Flavouring agent | 15 |
| 9. | Saccharine | 15 |
| 10. | Purified water | q.s |

Manufacturing Process:
1. Required quantity of the medium chain triglyceride was taken in a suitable vessel and heated to 65 to 70° C. Butylated hydroxy toluene was added followed by Abiraterone acetate and temperature was maintained till abiraterone was completely dissolved. (Oil phase)
2. Sodium metabisulfate, sorbic acid, polysorbate 80, EDTA, saccharine and flavoring agent were added to required quantity of water (aqueous phase) and heated to 65 to 70° C. till all ingredients were dissolved.
3. The oil phase was added to aqueous phase with simultaneous homogenization at 5000 rpm using high shear homogenizer till a coarse emulsion was obtained.
4. The coarse emulsion was further homogenized at 5000 psi over high pressure homogenizer for 3 passes maintaining the product temperature constant at 65 to 70° C.

Particle size analysis was performed for the nano-emulsion formulation prepared according to the example 3. Particle size data is summarized in table 4.

Table 4: Particle size data for the nano-emulsion formulation prepared according to the example 3.

| Parameter | Result |
|---|---|
| Z Average (nm) | 202.72 |
| D 10 (nm) | 118.06 |
| D 50 (nm) | 196.0 |
| D 90 (nm) | 325.88 |
| Poly dispersity index (%) | 18 |

### Example 4

### Nano-Suspension Formulation

| S. No | Ingredient | Quantity (mg) |
|---|---|---|
| 1 | Abiraterone acetate | 1000 |
| 2 | Medium chain triglyceride | 1000 |
| 3 | Polyvinyl pyrrolidone | 40 |
| 4 | Carbopol | 10 |
| 5 | Vanilla | 16 |
| 6 | Saccharine | 8 |
| 7 | Sodium lauryl sulphate | 45 |
| 8 | Vitamin E-TPGS | 25 |
| 9 | Sodium hydroxide | q.s |
| 10 | Water | q.s |

Manufacturing Process:
1. Polyvinyl pyrrolidone (PVP), sodium lauryl sulphate, saccharin and vanilla flavor were added to some amount of water and stirred (Phase A).
2. Vitamin E-TPGS was added to some amount of water, then it was heated with simultaneous stirring till vitamin E-TPGS was completely dissolved. (Phase B)
3. Abiraterone acetate was added to medium chain triglyceride and stirred (Phase C).
4. Carbopol was added to required quantity of water and stirred till a clear dispersion was obtained. Sodium hydroxide was added and stirred till the fluid becomes thick as a transparent and translucent gel (Phase D).
5. Phase A was transferred to phase B with stirring till a homogenous mixture was obtained.
6. Homogenous mixture obtained in step 5, was added to phase C with high speed stirring till the phase becomes uniform coarse dispersion.
7. The above coarse dispersion of step 6 was transferred to phase D with homogenization. The product was homogenized at 5000 to 6000 rpm for 10 minutes using high shear homogenizer.
8. The above dispersion was homogenized at 5000 psi using high pressure homogenizer for pass 1, and 7500 psi for pass 2 and 10000 psi for pass 3.

### Example 5

### Nano-Emulsion Formulation

| S. No | Ingredients | Quantity in mg |
|---|---|---|
| 1. | Abiraterone acetate | 500 |
| 2. | Medium chain triglyceride | 4000 |
| | (Labrafac lipophile WL 1349) | |
| 3. | Butylated hydroxy toluene | 3.75 |
| 4. | Sodium metabisulfate | 15 |
| 5. | Sorbic acid | 12 |
| 6. | Polysorbate 80 | 240 |
| 7. | Lecithin | 50-500 |
| 8. | EDTA | 7.5 |
| 9. | Flavouring agent | 15 |
| 10. | Saccharine | 15 |
| 11. | Purified water | q.s |

Manufacturing Process:
1. Required quantity of the medium chain triglyceride was taken in a suitable vessel and heated to 65 to 70° C. Butylated hydroxy toluene was added followed by Abiraterone acetate and temperature was maintained till abiraterone was completely dissolved. (Oil phase)
2. Sodium metabisulfate, sorbic acid, polysorbate 80, EDTA, saccharine, flavoring agent and lecithin were added to required quantity of water (aqueous phase) and heated to 65 to 70° C. till all ingredients were dissolved.
3. The oil phase was added to aqueous phase with simultaneous homogenization at 5000 rpm using high shear homogenizer till a coarse emulsion was obtained.
4. The coarse emulsion was further homogenized at 5000 psi over high pressure homogenizer for 3 passes maintaining the product temperature constant at 65 to 70° C.

### Example 6

Experiments were carried out to establish the surfactant concentration

| | Qty (% w/v) | | |
|---|---|---|---|
| | Formulation A | Formulation B | Formulation C |
| | Surfactant | Surfactant | Surfactant |
| | concentration | concentration | concentration |
| | (6.4% w/v) | (3.2% w/v) | (2.4% w/v) |
| | [75% | [75% | [75% |
| | Polysorbate-80 + | Polysorbate-80 + | Polysorbate-80 + |
| Ingredients | 25% Lecithin] | 25% Lecithin] | 25% Lecithin] |
| Abiraterone | 2.500 | 1.667 | 2.500 |
| Acetate | | | |
| Medium chain | 30.000 | 20.000 | 30.000 |
| triglycerides | | | |
| Butylated | 0.150 | 0.100 | 0.075 |
| hydroxy | | | |
| Toluene | | | |
| Sodium meta | 0.600 | 0.400 | 0.300 |
| bisulfate | | | |
| Sorbic acid | 0.120 | 0.080 | 0.060 |
| L-alpha | 1.600 | 0.800 | 1.800 |
| Lecithin | | | |
| granular | | | |
| Polysorbate 80 | 4.800 | 2.400 | 0.600 |
| Water | 59.855 | 74.303 | 64.478 |
| EDTA | 0.075 | 0.050 | 0.038 |
| Masking 2521 | 0.150 | 0.100 | 0.075 |
| Saccharine | 0.150 | 0.100 | 0.075 |
| | 100.0 | 100.00 | 100.00 |

TABLE 5
Stability data of Formulation A

| | | 1 M- | 3 M- | 6 M- | 1 M- | 3 M- | 6 M- | 1 M- | 3 M- | 6 M- |
|---|---|---|---|---|---|---|---|---|---|---|
| Parameter | Initial | 25° C. | 25° C. | 25° C. | 30° C. | 30° C. | 30° C. | 40° C. | 40° C. | 40° C. |
| Unknown imp | ND | 0.16 | 0.09 | 0.28 | 0.15 | 0.14 | 0.28 | 0.13 | 0.14 | 0.2 |
| Unknown imp | 0.08 | 0.09 | 0.09 | ND | 0.09 | 0.09 | ND | 0.08 | 0.1 | 0.13 |
| Unknown imp | 0.16 | 0.12 | 0.03 | ND | 0.13 | 0.03 | ND | 0.14 | 0.03 | 0.23 |
| Unknown imp | ND | ND | ND | ND | ND | ND | ND | ND | ND | ND |
| Unknown imp | ND | ND | ND | ND | ND | ND | ND | ND | ND | ND |
| Unknown imp | 0.15 | 0.14 | 0.15 | 0.08 | 0.11 | 0.07 | 0.04 | 0.09 | 0.06 | 0.02 |
| Unknown imp | 0.16 | ND | ND | ND | ND | ND | ND | ND | ND | 0.05 |
| Max unknown | 0.16 | 0.16 | 0.15 | 0.28 | 0.15 | 0.14 | 0.28 | 0.13 | 0.14 | 0.23 |
| Total imp | 0.91 | 1.07 | 1.23 | 1.62 | 1.14 | 1.41 | 2.03 | 1.43 | 2.43 | 4.74 |
| Assay | 104.6 | 105 | 104.5 | 104.2 | 104.8 | 103.0 | 103.3 | 104.4 | 103.7 | 101.6 |
| Globular size | 118.17 | 120.68 | 131.87 | — | 122.9 | 122.91 | | 123.14 | 124.98 | |
| (nm) | | | | | | | | | | |
| Polydisperse | 5.9 | 7.2 | 9.9 | — | 2.0 | 7.9 | | 9.7 | 4.3 | |
| index (%) | | | | | | | | | | |
| D10 (nm) | 78.698 | 80.527 | 85.97 | — | 81.24 | 80.94 | | 81.638 | 82.3 | |
| D50 (nm) | 108.986 | 108.321 | 118.18 | | 110.455 | 111.13 | | 113.643 | 113.63 | |
| D90 (nm) | 153.053 | 146.635 | 163.45 | — | 152.023 | 154.25 | | 159.208 | 158.47 | |
| Viscosity | 9.96 | 10.05 | 9.82 | — | 9.95 | 10.1 | | 11.3 | 9.87 | |
| (mpas) | | | | | | | | | | |
| Density | 0.994 | 0.995 | 0.996 | — | 0.994 | 0.996 | | 0.994 | 0.997 | |
| (g/cm³) | | | | | | | | | | |
| Zeta potential | −25.5 | −24.2 | −25 | — | −20.9 | −26.2 | | −24.2 | −24 | |
| (mV) | | | | | | | | | | |

TABLE 6
Stability data of Formulation B

| | | 1 M- | 3 M- | 6 M- | 1 M- | 3 M- | 6 M- | 1 M- | 3 M- | 6 M- |
|---|---|---|---|---|---|---|---|---|---|---|
| Parameter | Initial | 25° C. | 25° C. | 25° C. | 30° C. | 30° C. | 30° C. | 40° C. | 40° C. | 40° C. |
| Unknown imp | ND | 0.11 | 0.09 | 0.2 | 0.11 | 0.09 | 0.2 | 0.12 | 0.16 | 0.22 |
| Unknown imp | 0.08 | 0.1 | 0.1 | ND | 0.1 | 0.03 | ND | 0.1 | 0.12 | 0.01 |
| Unknown imp | 0.09 | 0.12 | 0.03 | ND | 0.12 | ND | ND | 0.14 | 0.03 | 0.15 |
| Unknown imp | 0.04 | ND | ND | ND | ND | ND | ND | ND | ND | ND |
| Unknown imp | ND | ND | ND | ND | ND | ND | ND | ND | ND | ND |
| Unknown imp | 0.17 | 0.14 | 0.17 | 0.12 | 0.15 | 0.15 | 0.12 | 0.17 | 0.13 | 0.02 |
| Unknown imp | 0.17 | ND | ND | ND | ND | ND | ND | ND | ND | 0.06 |
| Max unknown | 0.17 | 0.14 | 0.17 | 0.20 | 0.15 | 0.15 | 0.2 | 0.17 | 0.16 | 0.22 |
| Total imp | 1.01 | 1.03 | 1.41 | 1.87 | 1.12 | 1.46 | 2.02 | 1.57 | 2.74 | 5.22 |
| Assay | 107.6 | 106.8 | 104.3 | 104.8 | 106.7 | 104.4 | 105.3 | 105 | 104.6 | 102.6 |
| Globular size | 119.8 | 126.2 | 126.2 | | 125.7 | 129.4 | | 130.5 | 129.0 | |
| (nm) | | | | | | | | | | |
| Polydisperse | 12.1 | 8.2 | 6.8 | | 10.9 | 5.8 | | 3.7 | 9 | |
| index (%) | | | | | | | | | | |
| D10 (nm) | 79.607 | 83.30 | 83.85 | | 83.45 | 84.9 | | 86.61 | 85.51 | |
| D50 (nm) | 109.56 | 115.1 | 113.4 | | 114.6 | 117.5 | | 115.2 | 120.3 | |
| D90 (nm) | 152.33 | 160.4 | 155.75 | | 158.9 | 163.8 | | 154.8 | 172.2 | |
| Viscosity | 2.49 | 2.85 | 2.55 | | 2.72 | 2.79 | | 2.72 | 2.51 | |
| (mpas) | | | | | | | | | | |
| Density | 0.995 | 0.995 | 0.996 | | 0.995 | 0.997 | | 0.995 | 0.996 | |
| (g/cm³) | | | | | | | | | | |
| Zeta potential | −24.1 | −23.3 | −21.9 | | −23.9 | −25.1 | | −23.7 | −23.8 | |
| (mV) | | | | | | | | | | |

TABLE 7
Stability data of Formulation C

| | | 1 M- | 3 M- | 6 M- | 1 M- | 3 M- | 6 M- | 1 M- | 3 M- | 6 M- |
|---|---|---|---|---|---|---|---|---|---|---|
| Parameter | Initial | 25° C. | 25° C. | 25° C. | 30° C. | 30° C. | 30° C. | 40° C. | 40° C. | 40° C. |
| Unknown imp | 0.09 | 0.08 | 0.1 | 0.04 | 0.09 | 0.09 | 0.03 | 0.11 | 0.09 | 0.05 |
| Unknown imp | 0.07 | 0.03 | ND | 0.07 | 0.03 | ND | 0.07 | 0.03 | ND | 0.06 |
| Unknown imp | ND | ND | ND | ND | ND | ND | ND | ND | ND | ND |
| Unknown imp | ND | ND | ND | ND | ND | ND | ND | ND | ND | ND |
| Unknown imp | ND | ND | ND | 0.04 | ND | ND | 0.03 | ND | ND | 0.03 |
| Unknown imp | 0.21 | 0.19 | 0.16 | 0.11 | 0.23 | 0.13 | 0.09 | 0.21 | 0.08 | 0.04 |
| Unknown imp | 0.15 | 0.09 | ND | ND | 0.07 | ND | ND | ND | ND | ND |
| Max unknown | 0.21 | 0.19 | 0.16 | 0.11 | 0.23 | 0.13 | 0.09 | 0.21 | 0.09 | 0.06 |
| Total imp | 0.93 | 0.81 | 0.96 | 1.27 | 0.94 | 1.08 | 1.59 | 1.32 | 1.86 | 2.85 |
| Assay | 107.5 | 106.6 | 104.2 | 104.7 | 105.2 | 104.1 | 104.6 | 101.8 | 104.7 | 104.3 |
| Globular | 210.26 | 194.39 | 233.1 | | 200.05 | 207.76 | | 201.85 | 212.1 | |
| size (nm) | | | | | | | | | | |
| Polydisperse | 10.6 | 20.3 | 22.6 | | 10.9 | 10.5 | | 14 | 6.1 | |
| index (%) | | | | | | | | | | |
| D10 (nm) | 136.187 | 123.799 | 131.619 | | 123.868 | 130.195 | | 129.52 | 138.913 | |
| D50 (nm) | 184.426 | 170.924 | 222.353 | | 184.613 | 190.592 | | 182.229 | 187.526 | |
| D90 (nm) | 251.627 | 235.38 | 376.806 | | 275.172 | 279.329 | | 257.476 | 255.074 | |
| Viscosity | 5.07 | 4.99 | 4.11 | | 5.02 | 4.21 | | 4.78 | 4.72 | |
| (mpas) | | | | | | | | | | |
| Density | 0.987 | 0.987 | 0.989 | | 0.987 | 0.989 | | 0.988 | 0.988 | |
| (g/cm³) | | | | | | | | | | |
| Zeta potential | −16.6 | −21.0 | −24.7 | | −20.5 | −24.8 | | −20.1 | −21.8 | |
| (mV) | | | | | | | | | | |

These surfactant concentrations yielded satisfactory stability and globule size which was retained even at stress conditions.

Although the invention has been illustrated and described in greater detail with reference to the preferred exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. An oral emulsion formulation of abiraterone comprising:
(i) abiraterone,
(ii) one or more emulsifying agents,
(iii) an oil phase, and
(iv) an aqueous phase.

2. The oral emulsion formulation of claim 1, wherein abiraterone is dissolved or dispersed in the oil phase.

3. The oral emulsion formulation of claim 1, wherein the globule size is not more than 800 nm when stored at 40° C. for 3 months.

4. The oral emulsion formulation of claim 1, wherein the D50 of the globule size is not more than 400 nm when stored at 40° C. for 3 months.

5. The oral emulsion formulation of claim 1, wherein the one or more emulsifying agents are selected from the group comprising polysorbates, lecithin, sorbitan esters of fatty acids, and polyethoxylated oils.

6. The oral emulsion formulation of claim 5, wherein the concentration of the one or more emulsifying agents ranges from 1-10% w/v.

7. The oral emulsion formulation of claim 1, wherein the oils for the oil phase are selected from the group comprising medium chain triglycerides, soya bean oil, corn oil, safflower oil, sunflower oil, castor oil, medium chain fatty acids, and combinations thereof.

8. The oral emulsion formulation of claim 1, additionally comprising at least one of a flavoring agent, a coloring agent, a buffering agent, and a stabilizer.

9. An oral emulsion formulation of abiraterone comprising:
(i) abiraterone,
(ii) polysorbate-80,
(iii) lecithin,
(iv) an oil phase, and
(v) an aqueous phase.

10. The oral emulsion formulation of claim 9, wherein the concentration of polysorbate-80 and lecithin ranges from 1-10% w/v.
